(19) United States
(12) Patent Application Publication
(10) Pub. No.: US 2008/0138397 A1
(43) Pub. Date: Jun. 12, 2008

(54) PROCESSES FOR TASTE-MASKING OF INHALED FORMULATIONS

(75) Inventors: Jeffrey A. Schuster, Oakland, CA (US); David C. Cipolla, San Ramon, CA (US); Stephen Farr, Orinda, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: Aradigm Corporation

(21) Appl. No.: 11/876,407

(22) Filed: Oct. 22, 2007

Related U.S. Application Data
(60) Provisional application No. 60/862,751, filed on Oct. 24, 2006.

(51) Int. Cl. A61K 31/47; A61K 9/127; A61P 11/08

(52) U.S. Cl. 424/450; 514/313

(57) ABSTRACT

The present invention provides novel processes and methodologies to minimize the bitter or otherwise unpleasant taste, to minimize the tendency to stimulate the cough reflex, or to minimize oropharyngeal deposition of medically-active compounds administered by the pulmonary/inhalation route and to deliver hydroxychloroquine (HCQ) either singularly or in combination with an antimalarial and aminoquinolone by the pulmonary/inhalation route in a sustained release or other formulation that minimizes the bitter or otherwise unpleasant taste of HCQ or any potential to stimulate the cough reflex, and to deliver a dopaminergic compound or its prodrug, including ABT-431 by the pulmonary/inhalation route in a sustained release or other formulation that minimizes the unpleasant taste of the drug or any potential to stimulate the cough reflex, and to deliver a lantibiotic, including duramycin by the pulmonary/inhalation route in a sustained release or other formulation that minimizes the unpleasant taste of the drug or any potential to stimulate throat irritation.

### CROSS REFERENCE

This application claims the benefit of U.S. Provisional Application No. 60/862,751, filed Oct. 24, 2006, which application is incorporated herein by reference.

### FIELD OF THE INVENTION

The present invention relates to novel methodologies and processes for taste masking or reducing cough or irritation of formulations administered by pulmonary delivery.

### BACKGROUND OF THE INVENTION

The list of medically active compounds with a bitter or otherwise unpleasant taste is very long. Alkaloids, aspirin, omeprazole, ibuprofen, hydroxychloroquine (HCQ), chlorpheniramine maleate, pseudoephedrine, AZT, several protease inhibitors and metronidazole are only a few of the many therapeutic agents that are characterized by bitterness. Administration of such compounds has historically constituted a challenge, bitterness being closely linked to non-compliance with a prescribed regimen. Pediatric and geriatric populations, often likely to be restricted to liquid dosing, are especially impacted by bitter or unpleasant taste in medication. Examples of extremely bitter medications in pediatric therapeutics include combinations of sulfamethoxazole and trimethoprim, cefuoxime axetil, cephalexin, dexamethasone, docusate Na, erythromycin and its combination with sulfisoxazole, iron supplements, isoniazid, pediatric combinations of dextromethorphan, guaifenesin and pseudoephedrine, penicillin, phenobarbital, prednisone, trimethoprim, valproic acid, carbinoxamine maleate and vitamin supplements. Minimizing the bitterness of such compounds is an integral component of ensuring compliance with a treatment regimen.

Several drugs and excipients are known to cause irritation in the throat, and particularly to stimulate the cough reflex. Cough-stimulating or throat-irritating compounds include ACE inhibitors, chiral tetracyclic dopaminergic compounds, such as ABT-431, and excipients such as succinates, acetates, citrates, mannitol, and cationically-charged chemicals including cationic peptides, such as lantibiotics, and specifically duramycin. The pharmaceutical industry devises complex formulation methodologies to ensure that the dosage regimen or bioavailability of medically-active compounds taken orally is not impeded by their unpleasant taste or their tendency to stimulate the cough reflex.

For oral administration of bitter or cough-causing compounds, there exists a lengthy history of innovative solutions, from the relatively simple and proverbial sugar coating to complex techniques falling within three broad areas: masking with flavored additives such as sweeteners, flavorants or lipids (Among others, U.S. Pat. No. 6,770,296 and U.S. Pat. No. 7,101,572); masking by physical methods such as polymer coatings (Among others, U.S. Pat. No. 5,728,403; U.S. Pat. No. 6,663,893 and U.S. Pat. No. 6,270,807) and microencapsulation (Among others U.S. Pat. No. 6,139,865 and U.S. Pat. No. 5,814,332); and masking by chemical methods such as formation of inclusion complexes (e.g., U.S. Pat. No. 5,024,997). A newer methodology utilizes a molecular biology approach, by which a formulation can potentially include a bitter blocker—a compound that inhibits the bitter taste pathway. (U.S. Pat. No. 6,942,874).

Unpleasant taste, including bitterness, is not restricted to drugs taken by the oral route. Several medically-active agents requiring pulmonary delivery, including hydroxychloroquine (HCQ), corticosteroids like flunisolide, ipratropium bromide and nedocromil suffer from similar drawbacks. In this case too, masking the unpleasant taste or diminishing the medication's tendency to induce cough can enhance both availability and conformance to a dosage regimen. In contrast to the field of oral delivery, there exists no specific solution, formulations or methodologies for pulmonary delivery that have been devised to minimize bitter or otherwise unpleasant taste or to suppress or diminish cough. Some aspects of the inventions detailed in this application specifically address this formulation and methodology void. Masking of bitter or otherwise unpleasant taste of, or minimizing cough caused by, pulmonary formulations employing novel methodologies and processes and applicability of these novel methodologies and processes to chemical compounds including hydroxychloroquine and its combinations are disclosed in this application.

Currently, there are few synthetic or natural polymeric materials which can be used for the controlled delivery of drugs, including peptide and protein drugs, because of the strict regulatory compliance requirements, such as biocompatibility, clearly defined degradation pathway, and safety of the degradation products. The most widely investigated and advanced biodegradable polymers in regard to available toxicological and clinical data are the aliphatic poly(alpha.-hydroxy acids), such as poly(D,L- or L-lactic acid) (PLA) and poly(glycolic acid) (PGA) and their copolymers (PLGA). These polymers are commercially available and are presently being used as bioresorbable sutures. An FDA-approved system for controlled release of leuprolide acetate, the Lupron Depot.®, is also based on PLGA copolymers.

A. S. Sawhney and J. A. Hubbell, J. Biomed. Mat. Res., 24, 1197-1411 (1990), synthesized terpolymers of D,L-lactide, glycolide and c-caprolactone which degrade rapidly in vitro. The hydrophilicity of the material was increased by copolymerization with a poloxamer surfactant (Pluronic F-68). This poloxamer is a block copolymer comprising about 80% by weight of a relatively hydrophobic poly(oxypropylene) block and 20% by weight of a hydrophilic poly(oxyethylene) block. Copolymerization with the poloxamer resulted in a stronger and partly crystalline material which was mechanically stable at physiological temperatures (e.g. 37 degrees C.) in water.

One system, which can be fabricated in aqueous solution, is a class of block copolymers referenced above and marketed under the Pluronic™ tradename. These copolymers are composed of two different polymer blocks, i.e. hydrophilic poly(oxyethylene) blocks and hydrophobic poly(oxypropylene) blocks to make up a triblock of poly(oxyethylene)-poly(oxypropylene)-poly(oxyethylene). The triblock copolymers absorb water to form gels which exhibit reverse thermal gelation behavior.

Churchill et al, U.S. Pat. Nos. 4,526,938 and 4,745,160 show copolymers that are either self-dispersible or can be made self-dispersible in aqueous solutions. These copolymers are ABA triblock or AB block copolymers composed of hydrophobic A-blocks, such as polylactide (PLA) or poly(lactide-co-glycolide)(PLGA), and hydrophilic B-blocks, such as polyethylene glycol (PEG) or polyvinyl pyrrolidone.

Dunn et al, in U.S. Pat. No. 5,324,519, disclose the composition of a liquid formulation of a thermoplastic polymer and a pharmaceutically acceptable organic solvent (Trade Name Atrigel). The composition is administered as a liquid to an implant site, whereupon the solvent diffuses or dissipates into the surrounding aqueous tissue fluids. The thermoplastic polymer is not soluble in these aqueous fluids so that it coagulates or solidifies to form a microporous solid or gelatinous matrix. The composition is a liquid formulation of a thermoset prepolymer or copolymer, preferably an acrylic ester-terminated biodegradable prepolymer, which is capable of cross-linking in situ to form a polymeric or copolymeric solid or gelatinous matrix.

In U.S. Pat. No. 6,117,949, Rathi et al., a water soluble biodegradable ABA- or BAB-type triblock polymer is disclosed that is made up of a major amount of a hydrophobic polymer made of a poly(lactide-co-glycolide) copolymer or poly(lactide) polymer as the A-blocks and a minor amount of a hydrophilic polyethylene glycol polymer B-block, that possesses reverse thermal gelation properties.

U.S. Pat. No. 5,980,948 describes a composition comprised of a product including a biologically active agent encapsulated in a matrix comprising a polyetherester copolymer, such as a polyethylene glycol terephthalate/polybutylene terephthalate copolymer. The polyetherester copolymer protects the biologically active agent (including proteins, peptides, and small drug molecules) from degradation or denaturation.

EP 1184032 describes a method for producing hydrogels, based on crystallization of dextran or derivatives thereof. These hydrogels find use in pharmaceutical, medical and biotechnological applications, e.g. as controlled release systems for the delivery of active ingredients in in vivo and in vitro applications. The hydrogels are produced by crystallization from an aqueous solution that is essentially free of organic solvents or crystallization enhancers.

EP 0842657 describes a two phase controlled release system containing dextran and polyethylene glycol. EP 0941068 describes a two phase dextran-containing controlled release system for proteins.

Phospholipid vehicles as drug delivery systems were rediscovered as “liposomes” in 1965 by Bangham [Bangham et al., J. Mol. Biol. 13 (1) (1965) 238-252]. In the early 90's, three products for intravenous injection entered the market: Ambisome® for the systemic fungal treatment, and two chemotherapeutic liposomal formulations (Doxil® and Daunosome®). Vasopressin entrapped in PEGylated long-circulating liposomes even remained bioactive one month after intravenous injection.

A new approach, rather than using unilamellar or multilamellar liposomes, is based on the DepoFoam™ system. These multivesicular liposomes (1-100 μm) contain multiple non-concentric internal aqueous compartments and lead to an increase in the encapsulation efficiency. After subcutaneous injection, the release of encapsulated peptide and protein was shown to be prolonged up to 7 days for DepoInsulin and up to 3 weeks for the DepoLeuprolide® formulation [Ye, Q et al., DepoFoam technology, J. Control. Rel. 64 (1-3) (2000), 155-166].

The company Novosom AG has patented a novel liposome-based depot system for proteins and peptides. The Cagicles® depots are produced by a two-step method: first, proteins are dissolved in an aqueous medium and then added to solutions of membrane-forming substances, which are selected such that the resulting membrane enters into a reversible mutual reaction with the protein. This mild-condition process enables the increase of the encapsulation rate over 30% of incorporated protein. Furthermore, a one-month sustained protein release was feasible after subcutaneous or intramuscular injection of the Cagicles® depots [Panzner, S., Novosom AG, Application No. 2000-EP11079, Patent No. WO 2001034115 (2000)]. These studies have proven the basic applicability of liposomes. The solubility benefits of liposomes are well known and reported.

U.S. Pat. No. 6,277,413 describes a biodegradable microsphere having a matrix, the matrix comprising at least one type of biodegradable polymer, and at least one type of lipid; and a physiologically active substance that is releasable from the biodegradable microsphere.

### SUMMARY OF THE INVENTION

The invention includes a formulation designed for intrapulmonary delivery which is comprised of pharmaceutically active drug and an excipient. The pharmaceutically active compound is a compound which, upon inhalation, results in an undesirable response such as unpleasant taste, throat irritation and/or coughing and the like. This pharmaceutically active drug is combined with an excipient material which reduces or eliminates the undesirable response. The excipient material preferably provides for controlled release of the pharmaceutically active drug after intrapulmonary delivery. The drug may be included within a liposome. The invention includes an aerosol formed from the formulation which aerosol is comprised of particles which have an aerodynamic diameter in a range of about 1 micron to less than about 10 microns and more preferably in a range of about 2 to 5 microns ±20%.

According to aspects of the invention, inhalation systems, products and formulations are provided with appropriate performance characteristics to avoid or minimize deposition in the oropharynx.

According to aspects of the invention, inhalation systems, products and formulations are provided to mask bitter or unpleasant tastes and/or minimize cough induction or throat irritation of pulmonary formulations by the addition of formulation excipients and/or by unique formulation including but not restricted to liposome encapsulation;

According to aspects of the invention, pulmonary formulations are provided that reduce interaction with taste receptors; and

According to aspects of the invention, pulmonary formulations and excipients are provided that extend dissolution rate or release rate of the pharmaceutically-active compound or formulation so that the level of receptor activation does not exceed the perception threshold.

Inhaled HCQ is being evaluated for treatment of asthma and other respiratory diseases. According to one aspect of the invention, a sustained release form of HCQ is provided, thereby prolonging interaction of HCQ with the desired receptors in the pulmonary tissue. Further, such a formulation may be used in combination with one or more of the above-mentioned aspects of the invention to reduce bitter or otherwise unpleasant taste or to minimize the tendency to stimulate the cough reflex.

According to aspects of the invention, a combination product of HCQ, an aminoquinolone or an antimalarial compound, may be formulated. Agents that could conceivably be embraced within such a formulation include, but are not restricted to, steroids, short-acting bronchodilators, muscarinic antagonists, xanthenes, leukotriene antagonists, tryptase inhibitors, cytokine modulators etc. Further, such a combination formulation may be devised in tandem with one or more of the above-mentioned inventions to reduce bitter or otherwise unpleasant taste or to minimize the tendency to stimulate the cough reflex.

It will be clear to one skilled in the art that the delivery of the technology could be completed with a wide array of technologies, including but not restricted to liquid spray or dry powder inhalation systems, nebulizers or metered-dose inhalers. In one embodiment, the aerosol may be generated by delivery through a single-use disposable nozzle array, as described in U.S. Pat. Nos. 6,845,216; 6,694,975; 6,629,526; 6,263,872; 6,131,570; 5,957,124 and 5,906,202 and which are incorporated herein by reference in their entirety.

According to aspects of the invention, formulations that minimize the exposure of the drug to taste and cough receptors or to the oropharynx following delivery may be used. By coating the aerosol particles with excipients that dissolve slowly in the aqueous environment (e.g., PLGA, polymers, etc.) or by coating the drug molecules with excipients that release the drug slowly (including but not restricted to liposomes or surfactants), even though the same amount of drug may still deposit in the oropharynx or near the receptors, the peak drug concentration that is available to bind to the receptors immediately after inhalation may be attenuated resulting in a reduction in bitter or unpleasant taste or other side effects such as stimulation of the cough reflex. Sodium ions (Na+) in sodium salts, among others, have shown the potential to reduce the bitterness of some compounds.

Alternately, or in combination with the above aspects of the invention, processes and methodologies that actively reduce interaction of inhaled formulations with taste receptors by means other than coating of formulation or excipients molecules may be used. Such methodologies include formulations containing specific compounds, including but not restricted to nucleotides, that block the bitter or otherwise unpleasant taste of pulmonary formulations.

Additionally, formulations may be devised that slow down or extend the dissolution or release rates of unpleasant-tasting or cough-creating components in the aqueous environment of the oropharynx, providing yet another means of minimizing bitter taste and cough creation of formulations to be delivered by the inhalation route.

Another aspect of the invention is to limit the amount of unpleasant taste or cough by controlling the size distribution and the velocity of the aerosol to limit the amount of oropharyngeal deposition.

Hydroxychloroquine or HCQ, known as a therapeutic for asthma and other respiratory diseases (U.S. Pat. No. 6,572,858), and administered by the inhalation route, is characterized by unpleasant taste. Formulation of HCQ, either singularly, or in combination, using one or more of the methods described above increases the therapeutic benefit of inhaled HCQ to treat asthma and other respiratory diseases. Following the human clinical trials of AERx-inhaled HCQ, we discovered in the pharmacokinetics blood sample analysis that HCQ was rapidly absorbed from the lung. Thus, it would be an improvement to develop a formulation that provides a sustained release of HCQ in the lung, prolonging interaction of HCQ with the desired receptors in the lung tissue. Formulation strategies include encapsulation in liposomes or use of polymeric microspheres or nanospheres that slowly degrade and release HCQ. Other formulation strategies outlined previously can also be envisioned for such a formulation.

Another compound, ABT-431, a chiral tetracyclic dopaminergic compound (U.S. Pat. No. 5,659,037) that converts to A-86929, a selective dopamine D-1 receptor agonist is purported to cause cough upon inhalation. This compound has demonstrated efficacy for the treatment of Parkinson's disease but oral administration is limited due to extensive clearance via first pass metabolism resulting in an oral bioavailability of less than 4%. Inhalation delivery is ideal in that it avoids the first pass metabolism clearance with rapid absorption in less than 10 minutes (Okumu F W et al, Pharm Res., Vol. 19, 7, 1009-1012 (2002)). However, the tolerability is poor, and the 2-3 hour half-life implies the need for multiple administration episodes each day.

An inhaled formulation which overcomes these challenges would provide significant benefit to patients. According to one aspect of the invention, a sustained release form of ABT-431 is provided, thereby prolonging release of ABT-431 in the pulmonary tissue, resulting in prolonged levels in the bloodstream and reducing the need for more frequent administrations. Further, such a formulation may be used in combination with one or more of the above-mentioned aspects of the invention to reduce any otherwise unpleasant taste or to minimize the tendency to stimulate the cough reflex. Formulation strategies include encapsulation in liposomes or use of polymeric microspheres or nanospheres that slowly degrade and release ABT-431 or its product, A-86929. Other formulation strategies outlined previously can also be envisioned for such a formulation.

Lantibiotics, a series of antimicrobial peptides, may cause irritation of the throat and upper airways following inhalation, as is known for cationic peptides with antimicrobial properties (Papagianni, M. 2003. Biotechnol Adv 21:465). None of these compounds are marketed but these peptides have the potential to treat a number of therapeutic conditions through actions against phospholipase A2 or angiotension converting enzyme (Cotter P. D. et al, 2005. Current Protein and Peptide Science, Vol. 6, 61-75). Some of these peptides are candidates for development as novel therapeutic agents and complements to conventional antibiotic therapy because, in contrast to conventional antibiotics, they do not appear to induce antibiotic resistance while they generally have a broad range of activity, are bacteriocidal as opposed to bacteriostatic and require a short contact time to induce killing. A number of naturally occurring peptides and their derivatives are being investigated as novel anti-infective therapies for conditions as diverse as oral mucositis, lung infections associated with cystic fibrosis (CF) and topical skin infections.

An inhaled formulation which overcomes these challenges would provide significant benefit to patients. According to one aspect of the invention, a sustained release form of a cationic peptide or lantibiotic is provided, thereby reducing the peak concentration of the therapeutic in the throat or lung. According to aspects of the invention, formulations that minimize the exposure of the drug to taste and cough receptors or to the oropharynx following delivery may be used. By coating the aerosol particles with excipients that dissolve slowly in the aqueous environment (e.g., PLGA, polymers, etc.) or by coating the drug molecules with excipients that release the drug slowly (including but not restricted to liposomes or surfactants), even though the same amount of drug may still deposit in the oropharynx or near the receptors or other non-specific sites, the peak drug concentration that is available to interact with these sites immediately after inhalation may be attenuated resulting in a reduction in irritation or unpleasant taste or other side effects. Formulation strategies include encapsulation in liposomes or use of polymeric microspheres or nanospheres that slowly degrade and release the cationic peptide or lantibiotic. Further, such a formulation may be used in combination with one or more of the above-mentioned aspects of the invention to reduce any otherwise unpleasant taste or irritation.

### DEFINITIONS

“Aerodynamic diameter” is the diameter of a particle with unit density that settles at the same velocity as the particle in question under the influence of gravity.

“Aerosol” means a suspension of particles in a gaseous medium, e.g., air. An “aqueous aerosol” is an aerosol formed from an aqueous solution (i.e., a solution containing water as a solvent).

“Chemical stability” refers to the stability of the drug compound itself. To be chemically stable, the chemical structure remains constant and doesn't degrade.

“Physical stability” refers to the drug staying in solution, as desired for the formulation. To be physically stable, the drug cannot denature or come out of solution or otherwise lose the integrity of the desired formulation.

“Functional stability” refers to the stability of the formulation when used in an aerosolization device. To have functional stability, good aerosol performance must be achieved consistently. The aerosol generated has the same attributes, e.g., consistent viable fraction throughout.

“Emitted dose” or “ED” is the amount of aerosolized particles of the active ingredient (e.g., recombinant human interferon alpha-2b) that is emitted from a drug delivery device. “Mean emitted dose” is an arithmetic average of the emitted doses released over a repetition of a plurality of deliveries under the same conditions.

“Fine particle fraction” or “FPF” is the fraction of particles in an emitted dose that are of a size capable of reaching the deep lung or alveolar membranes. Unless otherwise indicated, fine particle fraction is calculated herein as that fraction of the particles which are less than or equal to about 3.5 microns as measured by a Cascade Impactor, light scattering methods, phase Doppler particle sizing or other applicable methods.

“Fine particle dose” or “FPD” is the amount of the active ingredient that actually reaches the target zone (i.e., deep lung, alveolar membranes) and is a product of emitted dose and fine particle fraction (i.e., FPD=ED×FPF).

“Mass median aerodynamic diameter” or “MMAD” is the aerodynamic diameter of the particle where 50% of the aerosol mass is in larger particles and 50% of the aerosol mass is in smaller particles.

“Particle size distribution” or “PSD” is a description of the way the mass of the aerosol is distributed across the range of aerosol particle sizes.

“Dosage form” or “DF” is a container closure system that is used to hold a dose (or partial dose) of a formulation prior to aerosolizing it.

“Pharmacokinetics” or “PK” refers to the study and characterization of the time course of drug absorption, distribution, metabolism and excretion.

“Pharmacodynamics” or “PD” refers to the study and characterization of the biochemical and physiological response to drugs and their mechanism of action.

“Microbe free” refers to the formulation being rendered free from microorganisms by aseptically passing it through a sterilized microbial retentive filter membrane.

“System efficiency” is defined as the portion of the drug in the container-closure system that reaches the systemic circulation.

“Bioavailability” refers to the portion of the emitted or delivered or inhaled dose from the container-closure system that reaches the systemic circulation.

“HCQ” refers to Hydroxychloroquine, an antimalarial with proven efficacy for treating inflammatory conditions including pulmonary disease states.

“Liposomes” refer to microscopic vesicles, each consisting of an aqueous core enclosed in one or more phospholipid layers and used to convey vaccines, drugs, enzymes, or other substances to target cells or organs.

“Sustained release” refers to the gradual release of an active agent over a period of time, allowing for a sustained effect or prolonged action.

“Taste masking” refers to formulation techniques, methodologies or processes that mask or hide the bitter or otherwise unpleasant taste of a formulation.

### DETAILED DESCRIPTION AND PREFERRED EMBODIMENTS

Before the present formulations, methods and systems are described, it is to be understood that this invention is not limited to particular formulations described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one skilled in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a liposome encapsulation” includes a plurality of such liposome encapsulations and reference to “the delivery system” includes reference to one or more delivery systems and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

An aspect of the invention is a composition comprising liposomes wherein the liposomes are comprised of a biodegradable material which material is biocompatible and suitable for administration to the lungs of the human patient. The liposomes encapsulate HCQ in singular or combination form. The formulation may be aerosolized and create aerosolized particles which have a diameter in a range of from about 0.5 μm to about 12 μm. Many lipids can be utilized for such formulation, including but not limited to HSPC and sphingomyelin, preferably egg sphingomyelin.

The biocompatible, biodegradable material used in the formation of the liposomes may be lipids which may be particular combinations of lipids such as egg sphingomyelin and cholesterol as well as other lipid components described or referenced here.

The composition and/or aerosolized composition of the invention may be manufactured particularly for asthma and other respiratory diseases. The formulation may be aerosolized and inhaled into a patient's lungs and used to treat asthma and other respiratory diseases.

### Generation of Liposomes Containing HCQ and its Combinations

Currently marketed inhalation combination products include Advair Diskus (GSK product containing 100, 250 or 500 mcg of fluticasone propionate and 50 mcg of salmeterol) and a likely soon to be marketed product including Symbicort Turbuhaler (AstraZeneca product delivering 80, 160 or 320 mcg budesonide and 4.5 or 9 mcg of formoterol). However, it is believed that no currently marketed combination product or product in development includes HCQ, an aminoquinolone or an antimalarial compound. Thus we are proposing that an inhaled product containing HCQ, an aminoquinolone or antimalarial compound be combined with another drug or drugs to treat asthma or other respiratory diseases. The other drugs that may potentially be used with HCQ include short-acting bronchodilators (e.g., β2-adrenergic receptor agonists like albuterol or indacaterol), M3 muscarinic antagonists (e.g., ipatropium bromide), K+-channel openers, long-acting bronchodilators (e.g., formoterol, salmeterol), steroids (e.g., budesonide, fluticasone, triamcinolone, beclomethasone, ciclesonide, etc.), xanthines, leukotriene antagonists (e.g., montelukast sodium), phosphodiesterase 4 inhibitors, adenosine receptor antagonists, other miscellaneous anti-inflammatories (e.g., Syk kinase inhibitors (AVE-0950), tryptase inhibitors (AVE-8923 & AVE-5638), tachykinin antagonists (AVE-5883), inducible nitric oxide synthase inhibitors (GW-274150) and others), transcription factor decoys, TLR-9 agonists, antisense oligonucleotides, CGRP, lidocaine, inverse β2-agonists, anti-infective oxidative therapies, cytokine modulators (e.g., CCR3 receptor antagonists (GSK-766994, DPC-168, AZD-3778), TNF-α production inhibitors (LMP-160 & YS-TH2), and IL-4 antagonists (AVE-0309)), small molecule inhibitors of IgE, cell adhesion molecule (CAM) inhibitors, small molecules targeting the VLA4 receptor or integrin α4β1 (e.g., R-411, PS-460644, DW-908e, & CDP-323), immunomodulators including those that block T-cell signaling by inhibition of calcineurin (Tacrolimus), heparin neutralizers (Talactoferrin alfa), cytosolic PLA2 inhibitors (Efipladib) and others.

In one embodiment of the instant invention, a composition for treating asthma and respiratory diseases is provided by encapsulating HCQ and suitable combination drugs in liposomes. The liposomes may comprise a variety of lipids, including but not limited to HSPC, ESM, cholesterol, etc. In one embodiment, said liposomes comprise lipids such as egg sphingomyelin and cholesterol (ESM:CH). The liposomes are comprised of naturally-occurring, biocompatible and biodegradable materials. Liposomes are used to encapsulate biologically active materials for a variety of purposes. Having a variety of layers, sizes, surface charges and compositions, numerous procedures for liposomal preparation and for drug encapsulation within them have been developed, some of which have been scaled up to industrial levels. Liposomes can be designed to act as sustained release drug depots and, in certain applications, aid drug access across cell membranes.

Methods for making bioadhesive liposomes can be found, for example, in U.S. Pat. No. 5,401,511 which is incorporated herein by reference in its entirety along with the patents and publications cited therein which describe liposomes and methods of making liposomes. These methods can be applied to materials such as egg sphingomyelin and cholesterol (ESM:CH). In recent years, successful attempts have been made to bind different substances to liposomes. For example, binding of chymotrypsin to liposomes has been studied as a model for binding substances to liposomal surfaces. Recognizing substances, including antibodies, glycoproteins and lectins, have been bound to liposomal surfaces in an attempt to confer target specificity to the liposomes. The liposomes may incorporate polyethylene glycol (PEG) to increase circulation times.

The number and surface density of the discrete sites on the liposomal surfaces for covalent bonding are dictated by the liposome formulation and the liposome type. The liposomal surfaces also have sites for non-covalent association. Covalent binding is preferred as non-covalent binding might result in dissociation of the recognizing substances from the liposomes at the site of administration since the liposomes and the bioadhesive counterparts of the target site (that is, the bioadhesive matter) compete for the recognizing substances. Such dissociation would reverse the administered modified liposomes into regular, non-modified liposomes, thereby defeating the purpose of administration of the modified liposomes.

To form covalent conjugates of recognizing substances and liposomes, crosslinking reagents have been studied for effectiveness and biocompatibility. One such reagent is glutaraldehyde (GAD). Through the complex chemistry of crosslinking by GAD, linkage of the amine residues of the recognizing substances and liposomes may be established.

The crosslinking reagents can include glutaraldehyde (GAD) and a water soluble carbodiimide, preferably, 1-ethyl-3-(3-dimethylaminopropyl) carbodiimide (EDC). The recognizing substances include gelatin, collagen, and hyaluronic acid (HA). Following these methodologies, recognizing substances may be utilized as an adhesive or glue to attach the liposomes onto a target area, such as the pulmonary surface.

Thus, while not essential to the instant invention, the use of such bioadhesive liposomes, particularly those having hyaluronic acid as the bioadhesive ligand, will potentially increase residence time in pulmonary sites, and reduce mucociliary clearance and macrophage uptake. The liposome may be formed using a material such as egg sphingomyelin and cholesterol (ESM:CH) and such a liposome may be used to deliver a drug such as HCQ.

The liposomes of the invention may be multilamellar, or unilamellar. Multilamellar vesicles (MLV) are prepared according to techniques well known in the art.

Exemplary liposome compositions and methods of making them are disclosed in U.S. Pat. Nos. 6,890,555; 6,855,296; 6,770,291; 6,759,057; 6,623,671; 6,534,018; 6,355,267; 6,316,024; 6,221,385 and 6,197,333, and in Taylor, K M G and Farr, S J. Preparation of liposomes for pulmonary drug delivery (Chapter 11) In: Liposome Technology, Volume 1, (2nd edition) (Gregoriadis, G, ed.) CRC Press, Boca Raton, 1992, pp. 177-195, and Taylor, K M G and Farr, S J. Liposomes for pulmonary drug delivery. In: Liposomes: 21 years on. Drug Targeting and Delivery Series, Volume I (Florence, A T and Gregoraidis, G, eds.), Harwood Academic Publishers, London, London (1993) 95-110 all of which are incorporated herein by reference. The liposomes of the invention may be multilamellar, unilamellar, or any configuration known such as described in the above patents. The liposomes of the instant invention are preferably made from dipalmitoyl phosphatidylcholine, distearyl phosphatidyl choline, palmitoyl oleayl phosphatidylcholine, sphingomyelin and/or cholesterol, although it will be clear to one skilled in the art that any of a number of lipids could be used. In a preferred embodiment, the liposomes comprise egg sphingomyelin and cholesterol (ESM:CH). In general, the size of the liposomes generated is preferably about 0.01 μm to 5 μm, more preferably 0.1 to 1 μm, most preferably about 400 nm in diameter.

When the liposomes are delivered to the lung as an aerosol, the aerosol may preferably comprise particles of diameter 1 μm, to more preferably 12 μm or 2 μm to 6 μm.

### Pharmaceutical Formulation of HCQ-Containing Liposomes

In some embodiments, HCQ with other active ingredients such as aminoquinolones or antimalarials may be encapsulated together within the liposomes. Additionally, the formulation may contain encapsulated HCQ in combination with aminoquinolones, antimalarials or other active ingredients that are not encapsulated, or various combinations thereof.

Formulations of the invention may include liposomes containing HCQ and combination molecules, in combination with an amount of alveolar surfactant protein effective to enhance the transport of the liposomes across the pulmonary surface and into the circulatory system of the patient. U.S. Pat. No. 5,006,343, issued Apr. 9, 1991, which is incorporated herein by reference, disclosed liposomes and formulations of liposomes used in intrapulmonary delivery. The formulations and methodology disclosed in U.S. Pat. No. 5,006,343 may be adapted for the application of HCQ and may be included within the delivery device of the present invention in order to provide for effective treatments of asthma and other respiratory diseases.

Regardless of the form of the drug formulation, it may be preferable to create particles for inhalation comprising liposomes in the range of about 0.5 μm to 12 μm or 2 μm to 6 μm or about 3-4 μm. By creating particles that have a relatively narrow range of size, it may be possible to further increase the efficiency of the drug delivery system and improve the repeatability of the dosing. Thus, it may be preferable that the particles not only have a size in the range of 0.5 μm to 12 μm or 2 μm to 6 μm or about 3-4 μm but that the mean particle size be within a narrow range so that 80% or more of the particles being delivered to a patient have a particle diameter which is within ±20% of the average particle size, preferably ±10% and more preferably ±5% of the average particle size.

It is known to those skilled in the art that the amount of oropharyngeal deposition, and thus the propensity for an aerosol to cause unpleasant taste or cough, is proportional to the particle size squared, the density of the particle, and the velocity of the particle. Thus, in addition to controlling the particle size, it is advantageous to control the particle velocity. This can be done in a number of ways, but is preferably achieved by implementing a system that limits the maximum inhaled velocity, in such a way that the inhaled velocity does not exceed a predetermined velocity, largely independently of the inspiratory effort of the subject. In this way oropharyngeal deposition may be limited to less than 25% of the inhaled aerosol, preferably less than 20%, more preferably less than 15%.

The formulations of the invention are preferably administered to a patient using a disposable package and portable, hand-held, battery-powered device, such as the AERx device described in U.S. Pat. No. 5,823,178, issued to Aradigm of Hayward, Calif. Alternatively, the formulations of the instant invention may be carried out using a mechanical (non-electronic) device. Other devices may be used, such as a nebulizer, including jet nebulizers, ultrasonic nebulizers, vibrating mesh nebulizers, a condensation aerosol generator, an electro-hydrodynamic nebulizer, a metered dose inhaler (MDI), drug powder inhaler (DPI) or other pulmonary delivery device known to those skilled in the art. The formulations could be repeatably delivered by permitting inhalation only at substantially the same inspiratory flow rate and inspired volume for each delivery.

An aerosol may be created by forcing drug through pores of a membrane which pores have a size in the range of about 0.25 to 6 microns, (see U.S. Pat. No. 5,823,178) and preferably with the pores ranging from 0.4 to 6 microns, more preferably from 0.4 to 1.2 microns. When the pores are of this size the particles which are formed when the formulation flows through the pores may have a diameter in the range of 0.25 to 12 microns. Drug particles may be released with an airflow intended to keep the particles within this size range. The creation of small particles may be facilitated by the use of the vibration device which provides a vibration frequency in the range of about 800 to about 4000 kilohertz. Those skilled in the art will recognize that some adjustments can be made in the parameters such as the size of the pores from which drug is released, vibration frequency, pressure, and other parameters based on the density and viscosity of the formulation keeping in mind that an object of one embodiment of the invention is to provide aerosolized particles having a diameter in the range of about 0.5 to 12 microns.

The liposome formulation may be a low viscosity liquid formulation. The viscosity of the drug by itself or in combination with a carrier should be sufficiently low so that the formulation can be forced out of openings to form an aerosol, e.g., using 200 to 800 psi to form an aerosol, in one embodiment having a particle size in the range of about 0.5 to 12 microns.

In accordance with another formulation, the HCQ-containing liposomes are provided as a dry powder by itself, and are dispersed by the patient's inhalation air flow, or preferably by an active means such as an air jet, a vibrating means, or any other active dispersal mechanism that imparts energy to the powder. Liposomes containing HCQ can be spray dried and then delivered by inhalation as a dry powder, or suspended in a low boiling point propellant, as described in U.S. Pat. No. 4,895,719, which is incorporated herein by reference.

In another embodiment, it is feasible to create a dry powder inhalation wherein micronized HCQ and suitable combination compounds are adhered to the surface of a carrier particle the same being formulated into a capsulated formulation for use in an inhaler device, in which a capsule is filled with an inhalant formulation, as described in U.S. Pat. No. 7,022,311, which is incorporated herein by reference.

In another embodiment, the HCQ-containing liposomes are provided in a solution or suspension formulation. The suspending medium (carrier) can be any pharmaceutically acceptable liquid, but the preferred liquids are water, ethanol, perfluorocarbons, or combinations thereof. The most preferred carrier is water. Other formulations make it possible to produce aerosolized forms of HCQ-containing liposomes which can be inhaled and delivered to a patient via the intrapulmonary route. Specific information regarding example formulations which may be used in connection with aerosolized delivery devices are described within Remington's Pharmaceutical Sciences, A. R. Gennaro, editor, Mack Publishing Company.

In a preferred embodiment, a solution of active ingredient is encapsulated into liposomes consisting of hydrogenated soy phosphatidyl-choline (HSPC) (70.6 mg/mL), a semi-synthetic fully hydrogenated derivative of natural soy lecithin (SPC), and cholesterol (29.4 mg/mL). The lipid is organized in a bilayer, with an average particle size of 75 to 120 nm. The sterile suspension is suspended in an isotonic buffer (25 mM histidine, 145 mM NaCl at pH 6.0, 300 mOsm/kg) and administered by inhalation.

One embodiment of combination products with HCQ that may be preferred is to have a single device and one group of dosage forms with HCQ and another group of dosage forms with another compound. Alternatively, a group of strips with HCQ and another compound may be used. The advantage of this approach is that dosing frequency may be optimized for each compound independently, and yet only one device and one instruction set may be needed. For example, if the optimal dosing regimen for HCQ were weekly, and there were a long-acting beta agonist (LABA) that could be given as infrequently as twice weekly, you could have two types of dosage forms, one with a combination of the LABA and HCQ, and one with only the LABA, each of which is administered once weekly. Alternatively, if a loading dose of HCQ is required, followed by less frequent dosing, one could envisage strips with only HCQ that are given during the loading period, followed by strips containing a combination of HCQ with a LABA.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. An inhalation formulation, comprising:
a pharmaceutically active compound which causes an undesirable response chosen from unpleasant taste, throat irritation and cough when administered alone by pulmonary delivery; and
a controlled release component combined with the active compound,
wherein the addition of the controlled release component to the active compound substantially reduces the undesirable response, and wherein the active compound and the controlled release component combination is prepared in a form suitable for pulmonary delivery.

2. The formulation of claim 1, wherein the controlled release component comprises a liposome.

3. The formulation of claim 1, wherein the controlled release component is formulated to extend release rates of the pharmaceutically active compound or formulation so that a level of receptor activation does not exceed a perception threshold.

4. The inhalation formulation of claim 1, wherein the pharmaceutically active compound is hydroxychloroquine, or a pharmaceutically acceptable salt thereof, and the undesirable response is unpleasant taste.

5. The formulation of claim 1, further comprising: a second pharmaceutically active compound.

6. The inhalation formulation of claim 1, wherein the pharmaceutically active compound is a dopaminergic compound or its prodrug, including ABT-431, or a pharmaceutically acceptable salt thereof, and the undesirable response is cough.

7. The inhalation formulation of claim 1, wherein the pharmaceutically active compound is a lantibiotic, including duramycin, and the undesirable response is throat irritation.

8. An inhalation aerosol optimized to minimize a response chosen from unpleasant taste, irritation and cough, said inhalation aerosol comprising particles of controlled size and velocity, such that the amount of oro-pharyngeal deposition is less than 25% of the inhaled aerosol.

9. The inhalation aerosol of claim 8, wherein the amount of oropharyngeal deposition is less than 20%.

10. The inhalation aerosol of claim 8, wherein the amount of oropharyngeal deposition is less than 15%.

11. The inhalation aerosol of claim 8, further comprising: Hydroxychloroquine.

12. The inhalation aerosol of claim 8, further comprising a dopaminergic compound or its prodrug, including ABT-431.

13. The inhalation aerosol of claim 8, further comprising a lantibiotic, including duramycin.

14. The inhalation aerosol of claim 8, wherein the velocity is controlled by incorporating a component in a device used to generate the aerosol, said component having the property that the maximum velocity at which the patient can inhale is substantially limited.

15. An inhalation aerosol, comprising hydroxychloroquine, and a second active pharmaceutical ingredient.

16. The inhalation aerosol of claim 15, wherein the second active pharmaceutical ingredient is a bronchodilator.

17. The inhalation aerosol of claim 16, wherein the bronchodilator comprises a long acting beta-agonist chosen from: formoterol, salmeterol.

18. A kit for the delivery of an aerosol to a subject in need thereof, the kit comprising: a device for the generation and delivery of aerosols, a first group of containers containing a first formulation, a second group of containers containing a second formulation.

19. The kit of claim 18, wherein the first formulation comprises hydroxychloroquine.

20. The kit of claim 18, wherein the second formulation comprises a bronchodilator.

21. The kit of claim 18, wherein the bronchodilator is a long acting beta agonist chosen from: formoterol, salmeterol.

22. The kit of claim 18, further comprising, a third group of containers containing a third formulation.

23. The kit of claim 22, wherein the third group of containers comprises one or more of: a short acting beta agonist, a steroid.

24. The kit of claim 18, wherein the first formulation and the second formulation comprise active ingredients chosen from short-acting bronchodilators (e.g., β2-adrenergic receptor agonists like albuterol or indacaterol), M3 muscarinic antagonists (e.g., ipatropium bromide), K+-channel openers, long-acting bronchodilators (e.g., formoterol, salmeterol), steroids (e.g., budesonide, fluticasone, triamcinolone, beclomethasone, ciclesonide, etc.), xanthines, leukotriene antagonists (e.g., montelukast sodium), phosphodiesterase 4 inhibitors, adenosine receptor antagonists, other miscellaneous anti-inflammatories (e.g., Syk kinase inhibitors (AVE-0950), tryptase inhibitors (AVE-8923 & AVE-5638), tachykinin antagonists (AVE-5883), inducible nitric oxide synthase inhibitors (GW-274150) and others), transcription factor decoys, TLR-9 agonists, antisense oligonucleotides, CGRP, lidocaine, inverse β2-agonists, anti-infective oxidative therapies, cytokine modulators (e.g., CCR3 receptor antagonists (GSK-766994, DPC-168, AZD-3778), TNF-α production inhibitors (LMP-160 & YS-TH2), and IL-4 antagonists (AVE-0309)), small molecule inhibitors of IgE, cell adhesion molecule (CAM) inhibitors, small molecules targeting the VLA4 receptor or integrin α4β1 (e.g., R-411, PS-460644, DW-908e, & CDP-323), immunomodulators including those that block T-cell signaling by inhibition of calcineurin (Tacrolimus), heparin neutralizers (Talactoferrin alfa), cytosolic PLA2 inhibitors (Efipladib), or combinations thereof.
